(19) United States
(12) Patent Application Publication
(10) Pub. No.: US 2022/0101181 A1
(43) Pub. Date: Mar. 31, 2022

(54) DETECTION OF ONSETS AND TERMINATIONS USING SPARSE CENSORED TIME SERIES

(71) Applicant: Palo Alto Research Center Incorporated, Palo Alto, CA (US)

(72) Inventors: Robert R. Price, Palo Alto, CA (US); Charlotte Price, Palo Alto, CA (US)

(21) Appl. No.: 17/034,914

(22) Filed: Sep. 28, 2020

(51) Int. Cl. G06N 20/00; G06N 7/00; G16H 70/60

(52) U.S. Cl. CPC G06N 20/00; G16H 70/60; G06N 7/005

(57) ABSTRACT

At least one activity history for a plurality of entities is received. The at least one activity history comprises at least two events. An inter-event gap distribution is learned using the at least one activity history for the plurality of entities. A current activity history for a current entity is received. A probability of at least one of an onset and a termination related to the current activity history is determined based on the learned inter-gap distribution. An output is produced based on the determined probability.

### TECHNICAL FIELD

The present disclosure is directed to implementing machine learning in real-world applications.

### BACKGROUND

Quantifying uncertainty about whether a process has an onset or termination in some sub-window may be difficult in situations in which sparse observations are made on a potentially longer process history. There are many applications where detecting onsets and terminations could be useful.

### SUMMARY

Embodiments described herein involve a method comprising receiving at least one activity history for a plurality of entities, the at least one activity history comprising at least two events. An inter-event gap distribution is learned using the at least one activity history for the plurality of entities. A current activity history for a current entity is received. A probability of at least one of an onset and a termination related to the current activity history is determined based on the learned inter-gap distribution. An output is produced based on the determined probability.

Embodiments involve a system comprising a processor and a memory storing computer program instructions which when executed by the processor cause the processor to perform operations. The operations comprise receiving at least one activity history for a plurality of entities, the at least one activity history comprising at least two events. An inter-event gap distribution is learned using the at least one activity history for the plurality of entities. A current activity history for a current entity is received. A probability of at least one of an onset and a termination related to the current activity history is determined based on the learned inter-gap distribution. An output is produced based on the determined probability.

A non-transitory computer readable medium storing computer program instructions for determining an answer to a question in a multi-party conversation, the computer program instructions when executed by a processor cause the processor to perform operations. The operations comprise receiving at least one activity history for a plurality of entities, the at least one activity history comprising at least two events. An inter-event gap distribution is learned using the at least one activity history for the plurality of entities. A current activity history for a current entity is received. A probability of at least one of an onset and a termination related to the current activity history is determined based on the learned inter-gap distribution. An output is produced based on the determined probability.

### BRIEF DESCRIPTION OF THE DRAWINGS

FIG. 1 shows an example of an observed sequence of events within an observation window in which the first observed event is assumed to be the onset in accordance with embodiments described herein;

FIG. 2 illustrates an example in which it cannot be determined whether the first observed event in the observation window is an onset in accordance with embodiments described herein;

FIG. 3 illustrates another example in which it cannot be determined whether the first observed event in the observation window is an onset in accordance with embodiments described herein;

FIG. 4 illustrates a way to model and predict the occurrence of events using datasets having sparse observations in accordance with embodiments described herein; and

FIG. 5 shows a block diagram of a system capable of implementing embodiments described herein.

The figures are not necessarily to scale. Like numbers used in the figures refer to like components. However, it will be understood that the use of a number to refer to a component in a given figure is not intended to limit the component in another figure labeled with the same number.

### DETAILED DESCRIPTION

Quantifying uncertainty about whether a process has an onset or termination in some sub-window may be difficult in situations in which sparse observations are made on a potentially longer process history. There are many applications where detecting onsets and terminations could be useful. Processes described herein can be used to build models of and make predictions about the onset or termination of processes. For example, one might want to learn which factors predict the onset of a disease. In the absence of labeled data, creating a relevant dataset involves a method of detecting when onsets and terminations have occurred. Embodiments described herein can be used directly in the detection for an application. For example, one might want to detect accounts that have been abandoned by a user and delete them to minimize security risks without inconveniencing users who have not actually left.

Detection of onsets and terminations is a fairly intuitive problem when the whole process history can be observed, but becomes difficult when only part of the process is observed and observed events are sparse. For example, in medical claims data, the time series starts when a patient starts coverage with an insurer, but the onset of a particular patient condition may occur before, after or cotemporally with the new coverage. Second, the time series may only contain observations when the patient actually interacts with the medical system so it is very sparse. In this setting a gap before the first event in a patient history may signal the onset of a condition or it might simply be due to the fact that the patient changed insurers.

Embodiments described herein involve quantifying uncertainty about onsets and terminations probabilistically. These probabilities can be thresholded to separate histories into those that have onsets and those that do not with a desired level of confidence. In some cases, one can use the probability of onset as a weight in a machine learning method that accepts observation weights. Embodiments described herein may work when events are very sparse, unlike change point detection, for example. Embodiments described herein can also make use of information across the entire population of histories to build good models, unlike change point detection which typically only makes use of data points in a single series. Finally, the method does not require an explicit event label be provided as the target for the prediction unlike change point detection or survival models. It is an unsupervised method. It can therefore be used to generate targets for supervised methods such as logistic regression.

Embodiments described herein use the internal gaps in the time series to learn the nominal distribution of gaps for the population. Internal gaps have a well-defined start and end because activity can be observed before and after them. The model is therefore based on clearly defined events. The initial or final interval can be tested against this distribution to compute the probability that the initial and/or final open interval comes from the same distribution as the internal intervals.

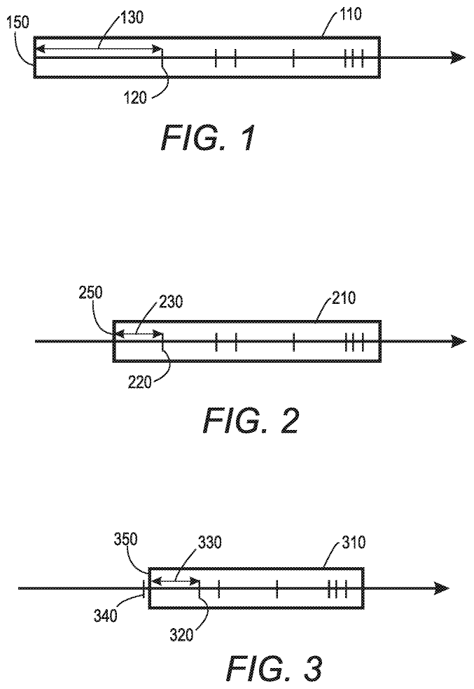

FIGS. 1-3 show three different examples of observed events within observation windows. In FIG. 1, the first observed event 120 within the observation window 110 can be assumed to be the onset. This is because there is a long time period 130 between the first observed event 120 and the start 150 of the observation window 110. Therefore, it is likely that the first observed event 120 is the onset. The onset for the example shown in FIG. 2 is less clear. This is because the first observed event 220 within the observation window 210 occurs a relatively short time period 230 after the start 250 of the observation window 210. In this case, it can be observed that the first observed event 220 within the observation window 210 is actually the onset because there are no preceding events before the start 250 of the observation window 210. FIG. 3 shows another example in which it is not clear whether the first observed event 320 within the observation window 310 is actually the onset because of the short time period 330 between the first observed event 320 within the observation window 310 and the start 350 of the observation window 310. In this case, it can be observed that the first observed event 320 within the observation window 310 is not the onset because there is a preceding event 340 before the start 350 of the observation window 310.

According to various embodiments, change point detection can be used to identify a time when properties of a process change. For example, one might be interested in the question of whether the rate of mining accidents changed during a certain time period. One could use statistical tests on the mean accident rate before and after a specific date to determine if there was a change at the date in question. The process can be repeated to check each possible date for its potential to be a change point. Change point algorithms typically return a list of dates when statistically significant changes have occurred.

The onset and termination detection problem described herein is a kind of change point problem where the change goes from no activity to positive activity or vice versa at the beginning or end of the sequence. Unfortunately, typical change point algorithms consider only one series at a time and have difficulty obtaining accurate statistical models of activity density especially when events are very sparse. For example, change point detection models may only work at a density greater than one event per time step but fail, for example, in health care claims where the sparsity is closer to 0.01 incidents per time step (i.e. per patient-day). Embodiments described herein make use of multiple histories of entities (e.g., patients, devices) to create a robust detection algorithm on vary sparse data. This allows for onset and termination algorithms that are much more robust than well-known change point algorithms on synthetic and realistic patient histories.

Hidden Markov Model analysis bears some similarity to change point algorithms. Instead of statistical tests, however, latent variables are used in a generative model to group observations into multiple states in order to maximize likelihood. It suffers similar problems to change point detection. It typically needs a lot of dense data samples to reliably assign data.

In hazard modelling or survival analysis, one can try to predict the time until some well-defined event. In medical clinical trials one might want to predict time until death. In industrial reliability models, one might want to predict time until failure of a component or system. The problem of onset or termination detection differs from traditional survival analysis because we may never get a definitive observation of an onset or termination. Instead, we may be given a window of observations and infer from this that an onset or termination has occurred. For example, one might see a cessation of email activity and hypothesize that an email account has been abandoned but there is no definitive ground truth that this has happened. Onset or termination detection could therefore be used to label sequences probabilistically as to where and when an event such abandonment may have occurred and this could be fed into a hazard model to model average time to abandonment. One could use a weighted hazard model which can exploit uncertainty estimates produced by the onset or termination detection algorithm.

In some cases, there are distinguishing patterns of observation attributes over time that can be used to predict the onset of an event. One can use a classifier, such as a recurrent neural network to recognize these spatio-temporal patterns. For example, one might use a Long short-term memory (LSTM) to detect the onset of notes in an audio recording of a musical performance. Like the hazard model, these methods predict the occurrence of the timing of an event given labeled training data. According to embodiments described herein, labeled training data is not available. As in the hazard model case, onset or termination detection could be used to label possible events and then methods such as LSTM could be used to predict these events from prior signals in the data.

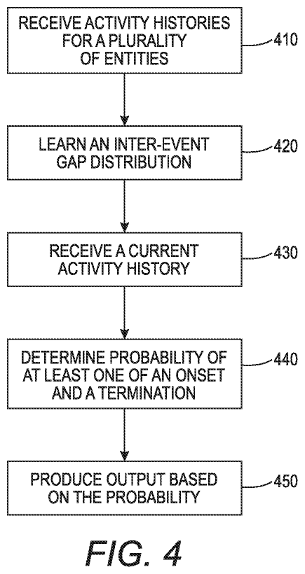

FIG. 4 illustrates a way to model and predict the occurrence of events using datasets having sparse observations in accordance with embodiments described herein. At least one activity history for a plurality of entities are received 410. According to various implementations, the at least one activity history comprises at least two events. In some cases, the entity comprises a patient and the current activity history comprises interactions between the patient and a healthcare entity. At least one of the onset and the termination may comprise at least one of the onset and a termination of a disease.

An inter-event gap distribution is learned 420 (e.g., using a machine learning process) using the at least one activity history for the plurality of entities. A current activity history for a current entity is received 430. According to various embodiments, the current activity history is at least one of sparse and censored.

A probability of at least one of an onset and a termination related to the current activity history is determined 440 based on the learned inter-gap distribution. In some cases additional information regarding the type of current activity history is received and the probability of at least one of the onset and the termination is determined based on the additional information.

The current activity history may comprise a first observed event and a last observed event. According to various implementations, it is determined if an elapsed time between the last observed event and a current time is greater than a predetermined threshold. The probability of the termination may be determined based on a determination that the elapsed time is greater than the predetermined threshold.

An output is produced 450 based on the determined probability. According to various implementations, it is determined whether the determined probability is greater than a predetermined threshold (e.g., 95%) and the output is produced based on the determination that the probability is greater than the predetermined threshold.

Embodiments described herein use an algorithm whose input is a set of histories and whose output is a probabilistic judgement for each history about whether an onset and/or termination occurred in each history. According to various implementations, it may be determined where the onset and/or termination occurred. While various methods described herein may be focused on detection of terminations, it is to be understood that the same or similar techniques can be used to detect onsets. Similarly, if techniques are described to detect onsets, it is to be understood that the same or similar techniques can be used to detect terminations. As an example, imagine that we are interested in predicting the termination event that occurs when an online game player quits playing the game. This cannot be observed directly, but it can be observed that the player has not been online for a predetermined period of time.

Z is defined as the observed duration of absence at the end of the sequence observation window. It might have been observed that the player has been offline for two weeks so far. Q can be defined as the unobserved event that a player has quit or not. A is defined as the actual duration of the player's absence (e.g., number of weeks a player will actually be absent). If the player has truly quit, this actual absence will be infinite in length as they are not coming back. If they have simply gone on vacation, they might be back in three weeks which will eventually be observed.

Embodiments described herein involve a way to infer the posterior probability of quitting using Bayes' rule, for example. Let Z be the observed duration of absence. Let Pr (Q|Z) be the probability of the event Q, that the player has quit, given an observed absence of duration Z. It can be expressed in terms of an unobserved actual absence duration A. Let Pr (Z|A) be the probability of seeing an observation of length Z given that the actual absence was of length A. Let Pr (A|Q) be probability of the actual absence being of length Z given that the player has quit or not. The posterior probability of quitting can be determined given the observation through Bayes' rule and the chain rule as follows in (1).

$$P r ( Q \vert Z ) = α P r ( Z \vert Q ) P r ( Q ) = α {\sum }_{A} P r ( Z \vert A ) P r ( A \vert Q ) P r ( Q )$$

The distribution of absence durations can be estimated when a player has not quit: Pr(A|Q̅). This can be done by looking for all those cases where the user's activity is not observed for some time but later returns over the entire population of users. If they returned, it can be determined that they did not quit. The distribution of absence durations can be determined when a player has quit: Pr(A|Q̅) as this is simply the distribution that puts all mass on the infinite duration (Pr(A=∞)=1) or a practical approximation (i.e., some very large duration) as the player is never returning. Finally, the prior probability of quitting Pr(Q) can be estimated. This can be set to an uninformative distribution Pr(Q)=0.5 for all values of Q or be derived from prior information. In some cases, additional statistical information can be used to provide a more accurate estimate. For example, in the case of employee quitting, estimates for turnover in specific industries can be used. For many industries, this is around ten percent a year.

The construction of Pr(A|Q̅) proceeds in several steps. The first step is to fit a density model for the inter-event durations. For discrete time series, a histogram over the set of integers can be used. For time series sampled at irregular intervals, parametric or nonparametric models can be used to capture the density. When time series are discrete time, the model F can simply be a histogram of integer absence durations.

When looking at the quitting probability αΣ_{A}Pr(Z|A)Pr(A|Q)Pr(Q), it can be observed that Pr (Z|A) defines an observation model which links the observed duration Z to the actual duration A. According to various configurations, the observed duration Z may differ from A in instances in which the observation sequence is censored. In the case of quitting, it may not be known how long A is until the absence is over and the user returns. Pr(Z|A) can be defined as shown in (2).

$$P r ( Z \vert A ) = \{ \begin{matrix}0 if Z < A \\ \frac{1}{{A}_{M A X}} if Z \geq A\end{matrix} \qquad ( 2 )$$

When we sum over possible absence durations, the complementary cumulative distribution is being calculated and/or the probability that Z could be greater or equal than some value. In some cases, it may be easier to build upon the cumulative distribution, that is, the probability that an absence duration is less than d. The cumulative distribution is built from the model that is constructed of inter-event densities (this becomes an integral in the continuous case) as shown in (3).

$$P r ( A \leq d \vert \overline{Q} ) = {\sum }_{i < d} P r ( A = i \vert \overline{Q} ) = {\sum }_{i < d} M ( i ) \qquad ( 3 )$$

The complementary cumulative is then just one minus the result from (3) as shown in (4).

$$C ( A ) = P r ( A \leq d \vert \overline{Q} ) = 1 - P r ( A \leq d \vert \overline{Q} ) = 1 - {\sum }_{i < d} P r ( A = i \vert \overline{Q} ) = 1 - {\sum }_{i < d} M ( i ) \qquad ( 4 )$$

The termination probability algorithm finds the last event in each series, computes the duration from this event to the current time T and assigns a termination probability using the complementary cumulative distribution, C, calculated earlier. The algorithm outputs a probability for each sequence of there being a quit event. Given a desired level of confidence, one can use these probabilities to classify them as actionable. For example, one could select those sequences where the probability of quitting is greater than 80% or 95% and assign them for follow-up emails or review by staff for possible account closure.

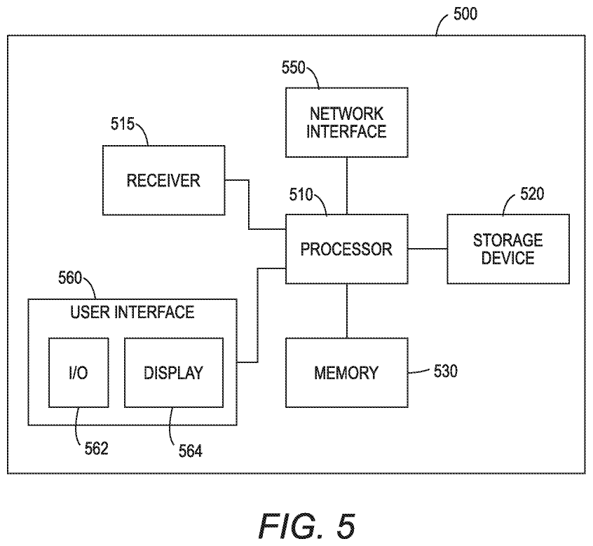

The above-described methods can be implemented on a computer using well-known computer processors, memory units, storage devices, computer software, and other components. A high-level block diagram of such a computer is illustrated in FIG. 5. Computer 500 contains a processor 510, which controls the overall operation of the computer 500 by executing computer program instructions which define such operation. It is to be understood that the processor 510 can include any type of device capable of executing instructions. For example, the processor 510 may include one or more of a central processing unit (CPU), a graphical processing unit (GPU), a field-programmable gate array (FPGA), and an application-specific integrated circuit (ASIC). The computer program instructions may be stored in a storage device 520 (e.g., magnetic disk) and loaded into memory 530 when execution of the computer program instructions is desired. Thus, the steps of the methods described herein may be defined by the computer program instructions stored in the memory 530 and controlled by the processor 510 executing the computer program instructions. The computer 500 may include one or more network interfaces 550 for communicating with other devices via a network. The computer 500 also includes a user interface 560 that enable user interaction with the computer 500. The user interface 560 may include I/O devices 562 (e.g., keyboard, mouse, speakers, buttons, etc.) to allow the user to interact with the computer. Such input/output devices 562 may be used in conjunction with a set of computer programs to receive visual input and display the human understandable output in accordance with embodiments described herein. The user interface also includes a display 564. The computer may also include a receiver 515 configured to receive visual input from the user interface 560 or from the storage device 520. According to various embodiments, FIG. 5 is a high-level representation of possible components of a computer for illustrative purposes and the computer may contain other components.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein. The use of numerical ranges by endpoints includes all numbers within that range (e.g. 1 to 5 includes 1, 1.5, 2, 2.75, 3, 3.80, 4, and 5) and any range within that range.

The various embodiments described above may be implemented using circuitry and/or software modules that interact to provide particular results. One of skill in the computing arts can readily implement such described functionality, either at a modular level or as a whole, using knowledge generally known in the art. For example, the flowcharts illustrated herein may be used to create computer-readable instructions/code for execution by a processor. Such instructions may be stored on a computer-readable medium and transferred to the processor for execution as is known in the art.

The foregoing description of the example embodiments have been presented for the purposes of illustration and description. It is not intended to be exhaustive or to limit the inventive concepts to the precise form disclosed. Many modifications and variations are possible in light of the above teachings. Any or all features of the disclosed embodiments can be applied individually or in any combination, not meant to be limiting but purely illustrative. It is intended that the scope be limited by the claims appended herein and not with the detailed description.

What is claimed is:

1. A method, comprising:
receiving at least one activity history for a plurality of entities, the at least one activity history comprising at least two events;
learning an inter-event gap distribution using the at least one activity history for the plurality of entities;
receiving a current activity history for a current entity;
determining a probability of at least one of an onset and a termination related to the current activity history based on the learned inter-gap distribution; and
producing an output based on the determined probability.

2. The method of claim 1, wherein the current activity history comprises a first observed event and a last observed event.

3. The method of claim 2, further comprising:
determining if an elapsed time between the last observed event and a current time is greater than a predetermined threshold; and
determining the probability of the termination based on a determination that the elapsed time is greater than the predetermined threshold.

4. The method of claim 1, further comprising:
determining whether the determined probability is greater than a predetermined threshold; and
producing the output based on the determination that the probability is greater than the predetermined threshold.

5. The method of claim 4, wherein the predetermined threshold is about 95%.

6. The method of claim 1, wherein the entity comprises a patient, the current activity history comprises interactions between the patient and a healthcare entity, at least one of the onset and the termination comprise at least one of the onset and a termination of a disease.

7. The method of claim 1, further comprising:
receiving additional information regarding the type of current activity history; and
determining a probability of at least one of the onset and the termination based on the additional information.

8. The method of claim 1, wherein the current activity history is at least one of sparse and censored.

9. The method of claim 1, further comprising learning the inter-event gap distribution using a machine learning process.

10. A system comprising:
a processor; and
a memory storing computer program instructions which when executed by the processor cause the processor to perform operations comprising:
receiving at least one activity history for a plurality of entities, the at least one activity history comprising at least two events;
learning an inter-event gap distribution using the at least one activity history for the plurality of entities;
receiving a current activity history for a current entity;
determining a probability of at least one of an onset and a termination related to the current activity history based on the learned inter-gap distribution; and
producing an output based on the determined probability.

11. The system of claim 10, wherein the current activity history comprises a first observed event and a last observed event.

12. The system of claim 11, further comprising:
determining if an elapsed between the last observed event and a current time is greater than a predetermined threshold; and
determining the probability of the termination based on a determination that the elapsed time is greater than the predetermined threshold.

13. The system of claim 10, further comprising:
determining whether the determined probability is greater than a predetermined threshold; and
producing the output based on the determination that the probability is greater than the predetermined threshold.

14. The system of claim 13, wherein the predetermined threshold is about 95%.

15. The system of claim 10, wherein the entity comprises a patient, the current activity history comprises interactions between the patient and a healthcare entity, at least one of the onset and the termination comprise at least one of the onset and a termination of a disease.

16. The system of claim 10, further comprising:
receiving additional information regarding the type of current activity history; and
determining a probability of at least one of the onset and the termination based on the additional information.

17. The system of claim 10, wherein the current activity history is at least one of sparse and censored.

18. The system of claim 10, further comprising learning the inter-event gap distribution using a machine learning process.

19. A non-transitory computer readable medium storing computer program instructions for determining an answer to a question in a multi-party conversation, the computer program instructions when executed by a processor cause the processor to perform operations comprising:
receiving at least one activity history for a plurality of entities, the at least one activity history comprising at least two events;
learning an inter-event gap distribution using the at least one activity history for the plurality of entities;
receiving a current activity history for a current entity;
determining a probability of at least one of an onset and a termination related to the current activity history based on the learned inter-gap distribution; and
producing an output based on the determined probability.

20. The non-transitory computer readable medium of claim 19, wherein the current activity history is at least one of sparse and censored.
